(19) United States
(12) Patent Application Publication
(10) Pub. No.: US 2012/0316121 A1
(43) Pub. Date: Dec. 13, 2012

(54) Materials and Methods for Modulating Arginine Metabolism

(76) Inventor: Ann de Wees Allen, St. Petersburg, FL (US)

(21) Appl. No.: 13/524,899

(22) Filed: Jun. 15, 2012

Related U.S. Application Data
(63) Continuation of application No. 12/652,902, filed on Jan. 6, 2010, now abandoned.
Application No. 12/652,902 is a continuation of application No. 11/494,898, filed on Jul. 28, 2006, now abandoned.
(60) Provisional application No. 60/703,765, filed on Jul. 28, 2005.

(51) Int. Cl. A61K 31/70; A61P 3/10; A61P 9/00; A61P 9/10; A61P 3/06; A61P 3/04

(52) U.S. Cl. 514/23

(57) ABSTRACT

The present invention provides novel compositions and methods for their production and use in modulating arginine metabolism to treat biological conditions. The biological condition is preferably one associated with dysregulated arginine metabolism and/or abnormal endogenous levels of substances resulting from or involved in arginine metabolism. The subject invention provides compositions that modulate levels of arginine or levels of substances that are derived from arginine in vivo via pre-selected signal transduction/metabolic pathways. In one embodiment, compositions comprising arginine and a glycoside are provided. The compositions and methods of the invention are able to select and prompt a particular metabolic pathway in which the arginine is to be used as a substrate.

### CROSS-REFERENCE TO A RELATED APPLICATION

This application is a continuation of U.S. patent application Ser. No. 12/652,902, filed Jan. 6, 2010; which is a continuation of U.S. patent application Ser. No. 11/494,898, filed Jul. 28, 2006; which claims the benefit of U.S. provisional application Serial No. 60/703,765, filed on Jul. 28, 2005, both of which are hereby incorporated by reference in their entirety, including any figures, tables, or drawings.

### BACKGROUND OF THE INVENTION

Many important metabolic pathways require arginine as a substrate (see, for example, Morris, S. M., Jr., “Enzymes of arginine metabolism,” J. Nutr. 134:2743S-2747S (2004) and Closs, E. et al., “Arginine Metabolism: Enzymology, Nutrition, and Clinical Significance,” J. Nutr., 134:2752S-2759S (2004)). Arginine is a versatile amino acid that gives rise to nitric oxide (NO), urea, ornithine, citrulline, creatine, agmatine, glutamate, proline, and polyamines (Wu, G. & Morris, S. M., Jr., “Arginine metabolism: nitric oxide and beyond,” Biochem. J. 336:1-17 (1998)). Arginine has also been shown to act as a signaling molecule that regulates essential cellular functions such as protein synthesis, apoptosis, and growth (see, for example, Yaman, I. et al., “The zipper model of translational control: a small upstream ORF is the switch that controls structural remodeling of an mRNA leader,” Cell 113:519-531 (2003)).

It is therefore no surprise that the metabolism of arginine is complex and highly regulated (Morris, S. M., Jr., “Regulation of enzymes of the urea cycle and arginine metabolism,” Annu. Rev. Nutr. 22:87-105. (2002)). This complexity arises not only from the diversity of enzymes involved in metabolism of arginine (L-arginine, in particular) and its metabolites but also from their cell-specific patterns of expression. Adding to the metabolic complexity is the fact that multiple isoforms exist for some of the enzymes involved in arginine metabolism (for example, there are at least 3 distinct isoforms of nitric oxide synthase (NOS) including neuronal NOS, inducible NOS (iNOS), and endothelial NOS) and 2 isoforms of arginase (types I and II; cytosolic and mitochondrial, respectively)). See, for example, Gloss, E. et al., “Arginine Metabolism: Enzymology, Nutrition, and Clinical Significance,” J. Nutr., 134:2752S-2759S (2004).

It has been proposed that abnormal levels of enzymes involved in arginine metabolism (such as arginase, an enzyme that converts arginine to ornithine and urea) and/or dysregulated arginine metabolism may contribute to various biological conditions. For example, various biological conditions have been linked with abnormal serum levels of organic compounds that are synthesized from arginine or whose production are stimulated or depressed by arginine, such as urea, ornithine, citrulline, creatine, agmatine, nitric oxide, glutamate, proline, hypothalamic corticotropin releasing factor (CRF), growth hormones (such as pituitary growth hormone and prolactin), pancreatic insulin, glucagon, pancreozymin and polypeptide, somatostatin, aldosterone, adrenal catecholamines, and/or polyamines. See, for example, Morris, C. et al., “Dysregulated arginine metabolism hemolysis-associated pulmonary hypertension, and mortality in sickle-cell disease,” JAMA, 294(1):81-90 (2005); and Reyes A A et al., “Role of arginine in health and in renal disease,” Am J. Physiol,267:F331-F346 (1994). Such biological conditions include, but are not limited to, stunted growth, paroxysmal nocturnal hemoglobinuria, erectile dysfunction, tumors, lupus erythematosus, rheumatoid arthritis, and pulmonary diseases.

For example, excessive production of arginase I and subsequent depletion of arginine has been observed in patients who present abnormal immune function. Exacerbation of arginase I production (as in uncontrolled autoimmune disease) can lead to two problems, a severe decrease in the immune response and the development of damaging scar tissue. See, for example, Ochoa, J. et al. “Arginase I Expression and Activity in Human Mononuclear Cells After Injury,” Ann of Surg, 233(3):393-399 (2001).

In certain instances, there is a relationship between dysregulated metabolism of arginine and/or abnormal levels of substances derived from arginine in vivo with genome variation. Millions of genetic profiles exist within the human genome. Genetic research has established that perturbations of the metabolic pathways involving organic substances can account for a large fraction of defects in humans, such as neural tube defects. For example, it has been hypothesized that the Arg(972) IRS-1 polymorphism may contribute to impaired endothelial function as well as the predisposition to develop cardiovascular disease and related symptoms, such as hypertension and insulin resistance.

In certain racial sectors, high-risk polymorphisms (genetic mutations) exist that cause life-threatening and life-shortening diseases. For example, the prevalence of vascular and endothelial disease is more prominently evidenced in the African American population as opposed to the Caucasian population. Endothelium-impaired function disorders, such as hypertension and diabetes mellitus, and the severity of their complications are considerably more severe among those of African descent than among Caucasians. Evidence has accumulated that elucidates the genetic connection between African Americans and vascular and cardiac pathophysiology.

Sickle cell disease is an inherited hemoglobulinopathy that causes the morphological distortion of red blood cells. Sickle cell disease includes sickle cell anemia, sickle-β-thalassemia, hemoglobin sickle cell disease, and hemoglobin C Harlem. Another inherited hemoglobulinopathy characterized by a structural hemoglobin defect is thalassemia. Thalassemia includes α-thalassemia and β-thalassemia (also known as Cooley's anemia, erythroblastic anemia, hereditary leptocytosis, and Mediterranean disease). These hereditary diseases have significant morbidity and mortality and affect individuals of African descent, as well as those of Mediterranean, Middle Eastern, and South East Asian descent. For example, β-thalassemia in particular affects individuals of Eastern descent.

The distortion of red cells that occurs with sickle cell disease and/or thalassemia often leads to two major physiological consequences: (1) chronic hemolytic anemia and (2) occlusion of small blood vessels that results in ischemic damage to tissues. The course of this disease is typically punctuated with a variety of painful crises called vaso-occlusive crises. These crises represent episodes of hypoxic injury and infarction in the organs, abdomen, chest, extremities, or joints. Leg ulcers are an additional manifestation of the vaso-occlusive tendency of this disease. Central nervous system involvement is common producing seizures and even strokes. Aplastic crises, also common, represent a temporary cessation of bone marrow activity and may be triggered by infections, folic acid deficiency, or both. Crises are normally episodic and reversible, but in certain instances they may be fatal. Damage from crisis episodes tends to be cumulative and, even in those individuals with milder forms of sickle cell disorders and/or thalassemia, life spans can be greatly reduced. Absent alternative intervention, patients typically die before the age of thirty.

Current methods for managing sickle cell disease, thalassemia, and/or any other hemoglobulinopathy include neonatal screening, advances in red cell transfusion medicine, iron chelation therapy, penicillin prophylaxis, hydroxyurea therapy, pharmaceuticals (such as zinc sulphate and sildenafil (commonly known as VIAGRA, Pfizer, New York, N.Y.), gaseous nitric oxide (NO), splenectromy, and stem cell transplantation (see, for example, Gladwin and Kato, “Cardiopulmonary Complications of Sickle Cell Disease: Role of Nitric Oxide and Hemolytic Anemia,” Hematology, 51-7 (2005); and Machado et al., “Sildenafil therapy in patients with sickle cell disease and pulmonary hypertension,” British J Haematology, 130(3):445 (2005)).

Despite the voluminous literature, little progress has been made in the treatment of these disabling disorders. For example, although hydroxyurea has been found to decrease the incidence of sickle cell pain crises, it has been observed to have a tendency to suppress the bone marrow's ability to make red blood cells, white blood cells, and platelets; and its long term safety is still unknown. Because of safety and efficacy issues, exchange transfusions and bone marrow transplantation are only utilized in severe cases or following ischemic stroke. The ideal treatment would involve replacement of the mutated genes with normal functional genes. Many laboratories are pursuing this goal; none have been successful (see, for example, Dover et al., “Hydroxyurea induction of hemoglobin F production in sickle cell disease: Relationship between cytotoxicity and F cell production,” Blood, 67:735-738 (1986); Delthia Ricks, “Gene therapy may cure 2 blood diseases,” Newsday (2006)). An alternative treatment would involve an oral, readily absorbed, non-toxic agent, capable of maximizing arginine and nitric oxide bioavailability in patients diagnosed with an inherited hemoglobulinopathy, without harming the patient. To date, no such agents have been identified.

Several other polymorphisms highly prevalent with African Americans include, but are not limited to, the 894T variant of a. specific gene, which typically manifests as high blood pressure; the CRTH2 gene in the peak linkage region on Chromosome 11, which is a strong candidate gene for asthma in African Americans; and the endothelial nitric oxide synthase (eNOS) gene, which appears to contribute to the development and progression of coronary artery disease (CAD). At this time, there are no drugs or dietary supplements that can adequately address the afore-mentioned polymorphisms as well as CAD risk factors in a coordinate or uniform manner.

L-arginine has been identified to provide certain general health benefits. For example, L-arginine has been identified as a vascular protectant that alleviates endothelial injury and/or corrects endothelial dysfunction. A study in which cholesterol-fed animals were supplied with a dietary supplement of L-arginine demonstrated a reduction in the extent of atheromatous lesions and restoration of endothelium-dependent arteriolar vasodilation (Cooke et al., “Antiatherogenic effects of L-arginine in the hypercholesterolemic rabbit,” J Clin Invest., 90(3):1168-72 (1992); and Kuo et al., “Pathophysiological consequences of atherosclerosis extend into the coronary microcirculation. Restoration of endothelium-dependent responses by L-arginine,” Circ Res., 70(3):465-76 (1992)).

In addition, the administration of L-arginine appears to provide a variety of health benefits, including cardiovascular benefits, such as lowering cholesterol and/or inhibiting platelet aggregation. Other benefits form the administration of L-arginine appear to include the treatment, prevention, and/or inhibition of heart disease and poor circulation. See e.g., U.S. Pat. Nos. 5,428,070; 5,385,940; 5.364,884; 5,217,997; 5,157,022; 5,032,608; 4,920,098; 4,420,432, and European Patent No. 0,546,796. Accordingly, it has been recommended that a person take 1-3 grams/day of arginine to help maintain an adequate supply of arginine in vivo and ensure proper metabolism.

Unfortunately, in independent clinical studies, the administration of free form L-arginine has been shown to cause serious side effects that negate its long-term use in humans (see, for example, Schulman et al., “L-Arginine Therapy in Acute Myocardial Infarction,” JAMA, 295(1):58-64 (January 2006); Takeuchi et al., “Direct detrimental effects of L-arginine upon ischemia--reperfusion injury to myocardium,” J. Mol. Cell Cardio., 27(7):1405-14 (1995); Mori et al., “Intra-coronary administration of L-arginine aggravates myocardial stunning through production of peroxynitrite in dogs,” Cardiovasc Resp, 40(1):113-23 (1998); and Kronon et al., “L-arginine, prostaglandin, and white cell filtration equally improve myocardial protection in stressed neonatal hearts,” J Thorac Cardiovasc Surg, 118(4):665-72 (1999)).

Supplementing a diet with arginine may stimulate nitric oxide (NO) production to toxic levels and induce hemodynamic instability with refractory hypotension (see, for example, Chen et al., “Effects of chronic treatment with L-arginine on atherosclerosis in apoE knockout mice and apoE/iNOS double knockout mice,” Arterio. Thromb. Vasc. Biol., 22:97-103 (2003). Oral use of L-arginine is not safe for long-term administration in humans, as all forms of orally ingestible L-arginine increase and stimulate the production of free radicals and peroxynitrite, and can induce improper metabolism of L-arginine to overproduce NO. Recent studies indicate that overproduction of NO can actually reduce sperm motility (in humans), thereby impairing fertility and the ability to reproduce. Other disadvantages arising from the administration of L-arginine include headache, diarrhea, flatulence, depletion of electrolytes, as well as depletion of vitamins, minerals, and the like. The side effects from the administration of L-arginine by itself vary significantly from individual to individual and run the gamut from mild to severe.

These side effects appear to be due to the human body's inability to appropriately select in which metabolic pathway (including enzyme isoforms) to apply L-arginine as a substrate. No one, to date, has identified an effective method for administering arginine, where the arginine is selectively applied to an appropriate metabolic pathway to treat a specific disease/disorder associated with inadequate endogenous arginine supply.

Moreover, as noted above, much remains to be learned about the complex and dynamic interrelationships between the arginine metabolic enzymes and how they relate to variations in nutritional requirements for L-arginine or in changes in biochemical markers in health and disease. Thus, there remains a need for new ideas for manipulating arginine metabolism by nutritional regimens as well as for effective treatment of genome-specific diseases/disorders.

### BRIEF SUMMARY OF THE INVENTION

The present invention provides novel compositions and methods for their production and use in modulating arginine levels in vivo via pre-selected signal transduction/metabolic pathways to treat biological conditions. Preferably, the subject invention provides unique compositions and methods for the treatment of biological conditions associated with dysregulated arginine metabolism.

Arginine (L-arginine in particular) can undergo numerous metabolic fates. In addition to its role as a component of most proteins, this amino acid can be converted to urea, L-citrulline, L-ornithine, L-proline, L-glutamate, and polyamines such as putrescine. Creatine, the high-energy phosphate storage form found in skeletal muscles, is also formed from L-arginine. Recently the decarboxylation of L-arginine via L-arginine decarboxylase to form agmatine has been reported. Agmatine may act as an endogenous antihypertensive agent, similar in mechanism to that of clonidine. Thus L-arginine plays an important role in the body's response to injury and other biological conditions.

In one embodiment, compositions comprising arginine and a glycoside are provided. The compositions and methods of the invention, unlike previously disclosed methods for modulating arginine levels in vivo, are able to select and prompt a particular metabolic pathway in which the arginine is to be used as a substrate. The selected metabolic pathway is preferably one associated with the synthesis of any one or a combination of the following compounds: urea, ornithine, citrulline, creatine, agmatine, nitric oxide, glutamate, proline, hypothalamic corticotropin releasing factor (CRF), growth hormones (such as pituitary growth hormone and prolactin), pancreatic insulin, glucagon, pancreozymin and polypeptide, somatostatin, aldosterone, adrenal catecholamines, and/or polyamines. In doing so, the compositions and methods of the invention are able to effectively modulate in vivo levels of arginine to correct dysregulated metabolic pathways in the treatment of biological conditions therefrom. Such biological conditions include, but are not limited to, dysfunctional wound healing, growth hormone deficiency, paroxysmal nocturnal hemoglobinuria, erectile dysfunction, tumors (such as breast cancer), lupus erythematosus, rheumatoid arthritis, renal diseases (such as ischemic renal failure and nephrosclerosis), cardiovascular and pulmonary diseases (such as atherosclerosis, pulmonary hypertension, vasodilatory impairment, intimal lesions in hypercholesterolemics, myointimal hyperplasia, and endothelial dysfunction). Advantageously, the compositions and methods of the invention confer therapeutic benefits without eliciting the detrimental side effects often observed when administering free form arginine.

In one embodiment, the compositions and methods of the invention advantageously address dysregulated arginine metabolism. According to the present invention, compositions and methods for the treatment and/or prevention of diseases/disorders associated with dysregulated arginine metabolism (including inherited polymorphic conditions such as thalassemia and sickle cell disease), or for preventing, delaying, and/or treating the development of complications associated with those diseases/disorders are provided. More specifically, the subject invention provides materials and methods for treating and/or preventing vascular, cardiovascular, and pulmonary diseases/disorders, or for preventing, delaying, and/or mitigating the development of vascular, cardiovascular, and pulmonary disease-related complications, through the concurrent administration of arginine and a glycoside to a patient.

Specifically exemplified herein is the use of arginine in combination with a glycoside to regulate arginine metabolism in the treatment of the diseases/disorders (and corresponding complications) including, but not limited to, diabetes (Types 1 and 2); chronic infectious diseases (such as Chlamydia, tuberculosis, leprosy, chronic active hepatitis, herpes, lupus erythematosus); vascular diseases, cardiovascular diseases, and pulmonary diseases (such as emphysema, chronic obstructive pulmonary disease, atherosclerosis, pulmonary hypertension, vasodilatory impairment, intimal lesions in hypercholesterolemics, myointimal hyperplasia, endothelial dysfunction, and asthma); psoriasis; erectile dysfunction; hypercholesterolemia; hyperinsulinemia; dysglycemia; hyperuricemia; high triglyceride levels (including high LDL levels); obesity, hyperglycemia; glucose intolerance; low HDL levels; dysfunctional wound healing; growth hormone deficiency; paroxysmal nocturnal hemoglobinuria; tumors (such as breast cancer); rheumatoid arthritis; and renal diseases (such as ischemic renal failure and nephrosclerosis).

In one embodiment, arginine is concurrently administered with a glycoside to a patient who has no observable symptoms of a biological condition associated with dysregulated arginine metabolism but has been determined to be susceptible to developing the biological condition (hereinafter such a patient is referred to as an “at-risk patient”). Methods for identifying at-risk patients for conditions associated with dysregulated arginine metabolism are well known in the art. For example, the detection of known biological markers associated with a biological condition can be used to identify at-risk patients. Additional factors that can be used, alone or in combination, to determine whether an at-risk patient is predisposed to developing a biological condition associated with dysregulated arginine metabolism include, without limitation, heredity (i.e., familial hypercholesterolemia), age, and sex (i.e., post-menopausal women over the age of 50).

In one embodiment, the compositions of the invention selectively prompt the use of arginine as a substrate in a particular arginine-dependent metabolic pathway to regulate nitric oxide production in the treatment of vascular and cardiovascular dysfunction. In a related embodiment, the compositions of the invention are administered to a patient to treat vascular and cardiovascular dysfunction associated with a polymorphism (such as polymorphisms in the endothelial cell nitric oxide synthase (ecNOS) gene and in the platelet GPIIIa PLA¹/A² gene). In a related embodiment, the compositions and methods of the subject invention provide novel methods for prompting the uptake of arginine in a particular metabolic pathway to treat diseases/disorders associated with race-specific polymorphisms (for example, to treat African American population for sickle cell anemia), gender-specific polymorphisms, and the like.

A therapeutically effective amount of arginine for administration to a patient can be from about 0.1 mg to 30 g per dose of arginine. Preferably, a daily dose of about 1 g to 60 g of arginine is administered to an adult patient. In a more preferred embodiment, about 5 to 10 g of L-arginine is administered with a glycoside 1 to 3 times daily to an adult patient.

A therapeutically effective amount of a glycoside to be concurrently administered with arginine can be from about 0.1 mg to 30 g per dose of administered arginine. Preferably, a daily dose of about 5 g to 60 g of a glycoside is concurrently administered with arginine to an adult patient. In a more preferred embodiment, about 5 to 10 g of TRUTINA DULCEM (see U.S. Patent Application Publication No. 20040022914) is concurrently administered with elemental L-arginine 1 to 3 times daily to an adult patient.

### BRIEF DESCRIPTION OF DRAWINGS

FIG. 1 shows a metabolic pathway of arginine.

FIG. 2 shows metabolic enzymes that use L-arginine as a substrate.

### DETAILED DISCLOSURE OF THE INVENTION

The present invention provides novel compositions and methods for their production and use in modulating arginine levels in vivo via pre-selected signal transduction/metabolic pathways to treat biological conditions. Preferably, the subject invention provides unique compositions and methods for the treatment of biological conditions associated with dysregulated arginine metabolism.

It is advantageous to define several terms before describing the invention. It should be appreciated that the following definitions are used throughout this application.

As used in the specification and in the claims, the singular form “a,” “an,” and “the” may include plural referents unless the context clearly dictates otherwise. Also, as used in the specification and in the claims, the term “comprising” may include the embodiments “consisting of” and “consisting essentially of.”

The term “dysregulated arginine metabolism,” as used herein, refers to abnormalities in arginine metabolic pathways as well as metabolic pathways associated with arginine metabolism. Metabolic pathways associated with arginine metabolism are well-known to the skilled artisan and can include, but are not limited to, ornithine metabolic pathway, citrulline metabolic pathway, urea metabolic pathway, and the like. Contemplated abnormalities include, but are not limited to, over-expression or under-expression of enzymes and isozymes necessary for arginine metabolism (such as arginase, nitric oxide synthase, and the like); over-synthesis or under-synthesis of substances derived from arginine (such as urea, creatine, nitric oxide, agmatine, and the like); and abnormal levels of endogenous arginine.

As used herein, reference to “arginine” includes 2-amino-5-guanidinovaleric acid, L-arginine free base, the various L-arginine salts, which includes pharmaceutically acceptable salts of L-arginine (such as 4-bisglyco-deuteroporphyrin L-arginate, 2,4-sulfonedeuteroporphyrin L-arginate, heme-L-arginate, and L-arginine hydrochloride), as well as prodrugs of L-arginine that can, for example, be readily metabolized in the body to produce L-arginine. Also included within the scope of the subject invention are pre-cursors, equivalents, analogs, derivatives, conjugates, and metabolites of arginine, which have the ability as, described herein to modulate arginine levels in vivo in the treatment of a biological condition associated with dysregulated arginine metabolism, prevention of such biological condition in an at-risk patient, or in the treatment of a complication associated with such biological condition.

Various pre-cursors, equivalents, analogs, derivatives, conjugates, and metabolites of arginine are well known and readily used by those skilled in the art and include, for example, alpha-ketoglutaric acid, glutamic acid, praline, N^{ω}-methyl-L-arginine, N^{ω}-amino-L-arginine, N^{ω}-nitro-L-arginine, and the like. As contemplated herein, the invention can utilize compounds that are known to enhance the endogenous production of arginine, including alpha-ketoglutaric acid, glutamic acid, or proline. Accordingly, in one embodiment of the subject invention, the advantages of arginine and a glycoside, as set forth herein, can be achieved by promoting the endogenous production of arginine through natural metabolic process such as through the administration of glutamic acid.

The term “pharmaceutically acceptable salt,” as used herein, refers to any salt of arginine is pharmaceutically acceptable and does not greatly reduce or inhibit the activity of arginine. Examples of suitable anions for the preparation of a pharmaceutically acceptable salt of arginine, in accordance with the subject invention, include bromide, fluoride, iodide, borate, hypobromite, hypochlorite, nitrite, nitrate, hyponitrite, sulfate, disulfate, sulfite, sulfonate, phosphate, diphosphate, phosphate, phosphonate, diphosphonate, perchlorate, perchlorite, oxalate, malonate, succinate, lactate, carbonate, bicarbonate, acetate, benzoate, citrate, tosylate, permanganate, manganate, propanolate, propanoate, ethandioate, butanoate, propoxide, chromate, dichromate, selenate, orthosilicate, metasilicte, pertechnetate, technetate, dimethanolate, dimethoxide, thiocyanate, cyanate, isocyanate, 1,4-cyclohexanedithiolate, oxidobutanoate, 3-sulfidocyclobutane-1-sulfonate, 2-(2-carboxylatoethyl)-cyclohexanecarboxylate, 2-amino-4-(methythio)-butanoate, and the like. The suitable cation for most salts is hydrogen. However, other cations such as sodium, potassium, and the like would be acceptable in the preparation of a salt of arginine for use in accordance with the subject invention.

The term “glycoside,” as used herein, refers to sugar derivatives. In certain embodiments, a glycoside is a water soluble compound that contains a carbohydrate portion (gylcone) and a non-carbohydrate portion (aglycone). Glycosides contemplated for use in accordance with the subject invention include, but are not limited to, glycosides that fall within the following categories: tannins, cardioactives, aldehydes, antrhaquinones, alcohols, saponins, lactones, cyanophores, isothiocyanates, isothiocyanates, phenols, and flavonals. The glycosides of the invention can be extracted from roots, leaves, plants, legumes, and fruit. Methods for obtaining glycosides from fruit are well known in the art and are described in, for example, U.S. Pat. Nos. 5,411,755; 4,084,010; 6,103,240; and 6,124,442. These methods generally include one or more extraction and/or concentration steps

Preferably, the compositions of the present invention contain glycosides derived from fruit. In certain embodiments, the glycosides of the present invention comprise triterpene and/or other terpene glycosides. Contemplated triterpene and terpene glycosides include the following: sweet diterpenoid glycosides compounds; ent-Kaurene type glycoside compounds; Dulcoside A, Rebaudioside A-E, Stevioside, Rubusoside, Suavioside A, B, G, H, I, J, and Steviol 13-O-O-D-glucoside (or Steviolmonoside), Labdane type glycoside compounds; Baiyunoside, Gaudichaudioside A, and Phlomisoside-I; Sweet Triterpenoid glycoside compound; Cycloartane glycosides; Abrusosides A-D; Oleanane glycosides type; Glycyrrhizin, Apioglycyrrhizin, Araboglycyrrhizin, and Periandrin 1-V; Cucurbitane glycosides; Siamenoside I, Mogroside W, V, and 11-Oxomogroside V; Secodammarane glycosides; Pterocaryosides A, B; Dammarane; and Gypenoside XX. Preferably, the compositions of the invention comprise arginine and TRUTINA DULCEM (a glycoside-containing composition disclosed in U.S. Patent Application Publication No. 20040022914).

The term “treatment” or any variation thereof (i.e., treat, treating, etc.), as used herein, refers to any treatment of a patient diagnosed with biological conditions associated with dysregulated arginine metabolism using the materials and/or methods of the invention. The term treatment, as used herein, includes: (i) preventing or delaying the presentation of symptoms of a biological condition associated with dysregulated arginine metabolism in an at-risk patient who has yet to display symptoms of the biological condition (such as sickle cell disease); (ii) ameliorating the symptoms associated with the biological, condition in a patient diagnosed with the biological condition (such as chronic anemia); (iii) preventing, delaying, or ameliorating the presentation of symptoms of a complication associated with the biological condition (i.e., vascular and cardiovascular diseases associated with sickle cell disease) in either an at-risk patient or a patient diagnosed with the biological condition; and/or (iv) relieving the biological condition (i.e. causing regression of sickle cell disease).

As used herein, the term “complication(s)” refers to a pathological process or event occurring during a disease or condition that is not an essential part of the disease or condition; where it may result from the disease/condition or from independent causes. For example, vascular-related complications refer to medical/clinical problems that occur more often in patients who suffer from vascular disease. For example, vascular-related complications include, without limitation, tissue death, gangrene, weakened blood vessels, strokes, blood clots, narrowed blood vessels, and pulmonary embolisms.

“Concurrent administration” and “concurrently administering,” as used herein, includes administering a compound or therapeutic method suitable for use with the compositions and methods of the invention in the modulation of biological factors to treat a biological condition associated with dysregulated arginine metabolism. According to the subject invention, arginine is concurrently administered with a glycoside to treat a biological condition associated with a polymorphism.

The term “effective amount,” as used herein, refers to the amount necessary to elicit the desired biological response. in accordance with the subject invention, the effective amount of arginine is the amount necessary to provide an observable effect in at least one biological factor (i.e., modulation of arginase activity or observable increase in nitric oxide levels) for use in treating a biological condition (such as modulating arginine levels to treat coronary artery disease, acute coronary syndromes, venous disease, platelet aggregation, myocardial infarction, and venous thromboembolism associated with polymorphisms in ecNOS gene and platelet GPIIIa PLA¹/A² gene). The effective amount may include the amount necessary to enable a 1% - 95% increase in serum levels of any one or combination of the following: nitric oxide, urea, ornithine, citrulline, creatine, agmatine, glutamate, proline, and polyamines.

In certain embodiments, the effective amount enables a 5%, 10%, 20%, 25%, 30%, 35%, 40%, 45%, 50%, 55%, 60%, 65%, 70%, 75%, 80%, 85%, 90%, 95%, 98%, 99% and 100% decrease in severity of complications associated with the genome-specific biological condition.

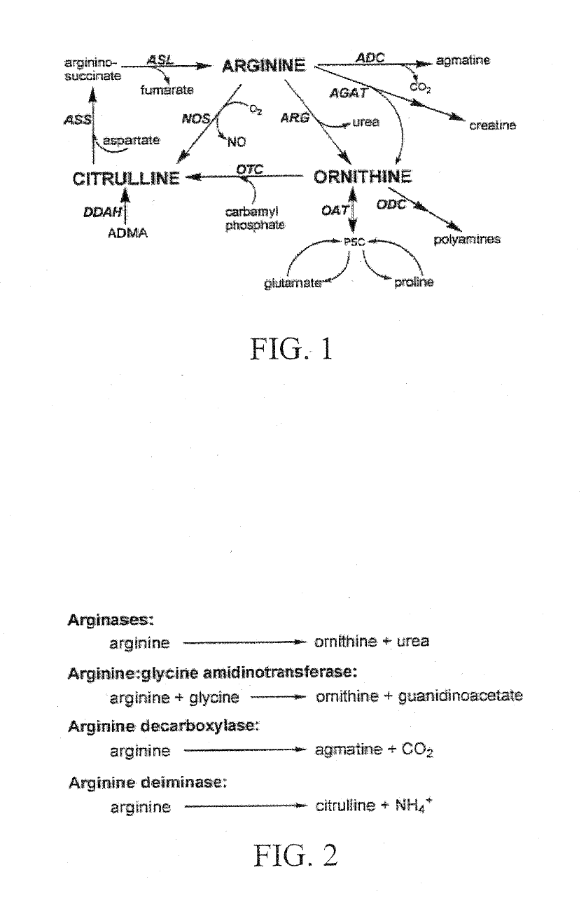

The subject invention provides compositions comprising arginine and a glycoside. The compositions and methods of the invention modulate arginine levels in vivo by selecting and prompting a particular metabolic pathway (FIG. 1) to use arginine as a substrate.

The selection and prompting of a particular metabolic pathway. according to the subject invention, includes the selection and prompting of specific enzymes and isozymes (FIG. 2) that: (a) catabolize arginine such as, but not limited to, arginases (such as types I and II; cytosolic and mitochondrial, respectively), nitric oxide synthases (such as neuronal NOS, inducible NOS (iNOS), and endothelial NOS), arginine:glycine amidinotransferase, arginine decarboxylase, dimethylarginine dimethylaminohydrolase (DDAH (EC3.5.3.18)), and arginine deiminase; and (b) synthesize arginine such as, but not limited to, argininosuccinate synthetase, and argininosuccinate lyase. See, for example, Morris, S., “Enzymes of Arginine Metabolism,” J. Nutr., 134:2743S-2747S (2004).

The selection and prompting of a particular metabolic pathway, according to the subject invention, also includes the selection and prompting of specific arginine transporters such as, but not limited to, cation amino acid transporters (such as CAT-1; CAT-2B; and CAT-3); heterodimeric amino acid transporters (such as the eight different HATs); and ATB^{0,+} (a glycoprotein that belongs to the SLC6 family, see Sloan, JL & Mager, S, “Cloning and functional expression of a human Na⁺ and Cl⁻-dependent neutral and cationic amino acid transporter B0+,” J. Biol. Chem., 274:23740-23745 (1999) and Closs, E et al., “Arginine Metabolism: Enzymology, Nutrition, and Clinical Significance,” J. Nutr., 134:2752S-2759S (2004)).

The selected metabolic pathway is preferably one associated with the synthesis of any one or combination of the following compounds: urea, ornithine, citrulline, creatine, agmatine, glutamate, nitric oxide, proline, and polyamines. In doing so, the compositions and methods of the invention are able to effectively modulate in vivo levels of arginine to correct dysregulated metabolic pathways in the treatment of diseases/disorders associated with polymorphisms. Advantageously, the compositions and methods of the invention confer therapeutic benefits without eliciting the detrimental side effects often observed when administering free form arginine.

More preferably, the compositions of the invention are administered to a patient to select and prompt a particular metabolic pathway. Compositions comprising L-arginine and a glycoside, preferably TRUTINA DULCEM, are administered to a patient to modulate nitric oxide synthesis in the treatment of biological conditions. In a related embodiment, compositions comprising L-arginine and a glycoside are administered to a patient to modulate nitric oxide synthesis in the treatment of biological conditions associated with one or more polymorphism linked to dysregulated arginine metabolism including, but not limited to, pulmonary hypertension; diabetes; neurological disorders; asthma; pyloric stenosis; atherosclerosis; stroke; coronary artery disease; high cholesterol; obesity; arrhythmia; systemic lupus erythematosus; renal dysfunction; nephrosclerosis; sickle cell disease; myocardial infarction; venous thromboembolism; embolisms; and other pulmonary, cardiac, vascular, or cardiovascular diseases.

In one embodiment, arginine is concurrently administered with a glycoside to a patient who has no observable symptoms of a biological condition associated with dysregulated arginine metabolism but has been determined to be susceptible to developing the biological condition (hereinafter such a patient is referred to as an “at-risk patient”). In a preferred embodiment, arginine is concurrently administered with a glycoside to a patient who has no observable symptoms of a biological condition associated with a polymorphism but is an “at-risk” patient for the polymorphism.

Methods for identifying at-risk patients for a biological condition associated with dysregulated arginine metabolism are well known in the art. The detection of known biological markers associated with a biological condition can be used to identify at-risk patients. For example, the abnormal levels of thrombomodulin, tissue factor pathway inhibitor, and soluble endothelium, leukocyte, and/or platelet selectins (sE-, sL-, sP-selectins, respectively) in plasma are known biomarkers of endothelial dysfunction. Additional factors that can be used, alone or in combination, to determine whether an at-risk patient is predisposed to developing a biological condition include, without limitation, heredity (i.e., familial hypercholesterolemia), age, and sex (i.e., post-menopausal women over the age of 50).

Moreover, methods for diagnosing various diseases associated with one or more polymorphisms are well-understood by the skilled clinician. For example, the skilled artisan would readily know how to detect sickle cell anemia (such as through a blood test and any one of several known methods for analyzing genes such as those listed above); hypertension (such as with a device that measures blood pressure and any one of several known methods for analyzing genes as listed above for detecting a polymorphism in the gene that expresses arginase or endothelial nitric oxide synthase); and the like.

In a preferred embodiment, the compositions of the invention selectively prompt the use of arginine as a substrate in a particular arginine-dependent metabolic pathway in the treatment of vascular and cardiovascular dysfunction associated with polymorphisms (such as polymorphisms in the endothelial cell nitric oxide synthase (ecNOS) gene and in the platelet GPIIIa PLA¹/A² gene). In a related embodiment, the compositions and methods of the subject invention provide novel methods for prompting the uptake of arginine in a particular metabolic pathway to treat diseases/disorders associated with race-specific polymorphisms (for example, to treat African American population for sickle cell anemia).

The subject invention preferably provides compositions comprising arginine and a glycoside in the treatment of one or more inherited polymorphic conditions associated with dysregulated arginine metabolism in any one or combination of the following genes; Arg(972) IRS-1; Ser128Arg; TP53; stromelysin-1 5A/6A; eNOS (such as eNOS polymorphism in T-786C in the 5′-flanking promoter region; eNOS4 with 27-bp repeat in intron 4; and G894T with Glu298Asp in exon 7); MTHFR C677T-A1298C; cardiac sodium channel gene (SCN5A); platelet GPIIIa PLA¹/A2; neuronal constitutive nitric oxide synthase gene (NOS1) and endothelial constitutive nitric oxide synthase (NOS3); endothelin-1 (EDN-1); CRTH2; Resistin-like molecule beta (RELM-β); β-globulin; and C825T.

The efficacy of treatment of a biological associated with dysregulated arginine metabolism, in accordance with the subject invention, can be measured by determining the ability of said treatment to (1) increase and/or decrease arginine levels in vivo; and/or (2) increase and/or decrease levels of urea, ornithine, citrulline, creatine, agmatine, nitric oxide, glutamate, proline, and/or polyamines in vivo. Measurement can be conducted on specific fluid cells, such as the cytoplasm of peripheral blood cells or monocyte/macrophages or lymphocytes or in serum. For example, according to the subject invention, the fluid of cells of interest are isolated from the patient and arginine (or other compound) levels can be measured through arginase enzyme activity. Other methods for measuring arginine (or other compound) levels in bodily fluids are well known to the skilled artisan.

The compositions of the invention can be used in a variety of routes of administration, including, for example, orally-administrable forms such as tablets, capsules or the like, or via parenteral, intravenous, intramuscular, transdermal, buccal, subcutaneous, suppository, or other route. Such compositions are referred to herein generically as “pharmaceutical compositions.” Typically, they can be in unit dosage form, namely, in physically discrete units suitable as unitary dosages for human consumption, each unit containing a predetermined quantity of active ingredient calculated to produce the desired therapeutic effect in association with one or more pharmaceutically acceptable other ingredients, i.e., diluent or carrier.

The compositions of the subject invention comprising arginine and a glycoside can be formulated according to known methods for preparing pharmaceutically useful compositions. Formulations are described in a number of sources, which are well known and readily available to those skilled in the art. For example, Remington's Pharmaceutical Science (Martin E W [1995] Easton Pennsylvania, Mack Publishing Company, 19^{th} ed.) describes formulations that can be used in connection with the subject invention. Formulations suitable for parenteral administration include, for example, aqueous sterile injection solutions, which may contain antioxidants, buffers, bacteriostats, and solutes, which render the formulation isotonic with the blood of the intended recipient; and aqueous and nonaqueous sterile suspensions, which may include suspending agents and thickening agents. The formulations may be presented in unit dose or multi-dose containers, for example sealed ampoules and vials, and may be stored in a freeze dried (lyophilized) condition requiring only the condition of the sterile liquid carrier, for example, water for injections, prior to use. Extemporaneous injection solutions and suspensions may be prepared from sterile powder, granules, tablets, etc. It should be understood that in addition to the ingredients particularly mentioned above, the formulations of the subject invention can include other agents conventional in the art having regard to the type of formulation in question.

In one embodiment, compositions of the invention comprise arginine, a glycoside, and a carrier such as inclusion compound host materials. It is believed that by providing a carrier such as inclusion compound host materials, stabilized arginine and glycoside molecules can be safely delivered to a patient at a dosage that will not induce toxicity. In addition, such carrier materials can include coating materials (i.e., enteric-coatings) that allow dissolution of the coating in an alkaline environment such as in the intestines.

Inclusion compound host materials that can be used in accordance with the subject invention include those disclosed in U.S. Patent Application No. 20040033985, incorporated herein in its entirety. Contemplated inclusion compound host materials include proteins (such as albumin), crown ethers, polyoxyalkylenes, polysiloxanes, zeolites, cholestyramine, colestipol, colesevelam, colestimide, sevelamer, cellulose derivatives, dextran derivatives, starch, starch derivatives, and pharmaceutically acceptable salts thereof. Contemplated cellulose derivatives and dextran derivatives include DEAF-cellulose, guanidinoethylcellulose, or DEAE-Sephadex. Favorable starches or starch derivatives to be included in the compositions of the invention include cyclodextrin, retrograded starch, degraded starch, a combination of retrograded and degraded starch, hydrophobic starch, amylase, starch-diethylaminoethylether, and starch-2-hydroxyethylether.

Concurrent administration of arginine and a glycoside, in accordance with the subject invention, can be accomplished by any suitable method and technique presently or prospectively known to those skilled in the art. In a preferred embodiment, arginine and a glycoside are formulated in a patentable and easily consumed oral formulation such as a pill, lozenge, tablet, gum, beverage, etc. The consumption is then taken at, prior to, or after, the diagnosis of a biological condition associated with a polymorphism.

In certain embodiments of the invention, a patient is assessed to identify the risk of developing a biological condition associated with associated with dysregulated arginine metabolism prior to the administration of a composition comprising arginine and a glycoside. Methods for identifying an at-risk patient are well known in the art. For example, contemplated methods for detecting polymorphisms associated with dysregulated arginine metabolism in accordance with the subject invention include, but are not limited to, those disclosed in U.S. Pat. Nos. 6,727,063; 6,525,185; 6,475,736; 6,340,566; 6,306,588; 6,120,992; 5,972,601; 5,780,229; and 5,604,099.

The precise amount of arginine and glycoside suitable for use in the practice of the present invention will vary depending on the adjuvants or synergists present, the size and kind of the patient, and specific form of arginine administered (such as a salt, pre-cursor, analog, etc.). A therapeutically effective amount of arginine for administration to a patient can be from about 0.1 mg to 30 g per dose of arginine. Preferably, a daily dose of about 1 g to 60 g of arginine is administered to an adult patient. In a more preferred embodiment, about 5 to 10 g of elemental L-arginine is administered with a glycoside 1 to 3 times daily to an adult patient.

While not being bound to any theory, the mechanism of selecting and prompting a particular metabolic pathway (including selecting and prompting a particular isozyme for arginine metabolism or an arginine transporter) to use arginine as a substrate is based upon the concurrent administration of a glycoside with arginine. It appears the glycoside component is involved in the signaling a particular metabolic pathway for processing the arginine.

According to the subject invention, the weight ratio of a glycoside to arginine in a composition of the invention can be any of the following (with glycoside:arginine): 0.25:1; 0.5:1; 0.75:1; 1:1; 1:0.25; 1:0.5; and 1:0.75. Accordingly, a therapeutically effective amount of a glycoside to be concurrently administered with arginine can be from about 0.1 mg to 30 g per dose of administered arginine. Preferably, a daily dose of about 5 g to 60 g of a glycoside is concurrently administered with arginine to an adult patient. In a more preferred embodiment, about 5 to 10 g of TRUTINA DULCEM (see U.S. Patent Application Publication No. 20040022914) is concurrently administered with elemental L-arginine 1 to 3 times daily to an adult patient.

The present application is also directed to a kit having at least one compartment, wherein a first compartment comprises a composition comprising an effective amount of arginine and a glycoside. In certain embodiments where the kit has more than one compartment, a second compartment includes at least one item appropriate for the treatment of a biological condition associated with one or more polymorphism.

The additional item, which would typically be separately compartmentalized within the kit, may be either instructional or therapeutic, or both. The additional item may be, for example, an item known to be therapeutically effective in the treatment of the biological condition of interest. Such items include, but are not limited to, analgesics, supplements, food items, and pharmaceutical compositions.

All patents, patent applications, provisional applications, and publications referred to or cited herein are incorporated by reference in their entirety, including all figures and tables, to the extent they are not inconsistent with the explicit teachings of the specification.

It should be understood that the examples and embodiments described herein are for illustrative purposes only and that various modifications or changes in light thereof will be suggested to persons skilled in the art and are to be included within the spirit and purview of this application and the scope of the appended claims.

I claim:

1. A method for treating a patient suffering from a condition associated with dysregulated arginine metabolism that comprises: diagnosing the condition; and
administering to said patient a composition comprising an effective amount of an arginine, or a pharmaceutically acceptable salt thereof; a glycoside; and a pharmaceutically acceptable carrier.

2. The method of claim 1, wherein the composition is orally consumed by the patient.

3. The method of claim 1, wherein the arginine is L-arginine and the glycoside is administered as a component of TRUTINA DULCEM.

4. The method of claim 3, wherein the effective amount of the L-arginine is about 0.1 to about 60 g daily, or an equivalent molar quantity.

5. The method of claim 1, wherein the condition is an inherited polymorphic condition.

6. The method of claim 5, wherein the inherited polymorphic condition is selected from the group consisting of: sickle cell anemia, sickle α-thalassemia, sickle β-thalassemia, hemoglobin sickle cell disease, hemoglobin C Harlem, α-thalasemia, and β-thalassemia.

7. A composition comprising an effective amount of an arginine, or pharmaceutically acceptable salt thereof; a glycoside; and a pharmaceutically acceptable carrier.

8. The composition of claim 7, which comprises a compound selected from the group consisting of: 2-amino-5-guanidinovaleric acid; L-arginine free base; 4-bisglyco-deuteroporphyrin L-arginate; 2,4-sulfonedeuteroporphyrin L-arginate; heme-L-arginate; L-arginine hydrochloride; L-arginine prodrug; alpha-ketoglutaric acid; glutamic acid; praline; N^{ω}-methyl-L-arginine; N^{ω}-amino-L-arginine; and N^{ω}-nitro-L-arginine.

9. The composition of claim 7, which comprises a compound selected from the group consisting of: tannins; cardioactives; aldehydes; antrhaquinones; alcohols; saponins; lactones; cyanophores; isothiocyanates; isothiocyanates; phenols; and flavonals.

10. The composition of claim 7, wherein the glycoside is a triterpene or a terpene.

11. The composition of claim 7, wherein the glycoside is TRUTINA DULCEM.

12. A. method for modulating arginine levels in vivo via pre-selected metabolic pathways, said method comprising: administering to a patient a composition comprising an effective amount of arginine and a glycoside compound.

13. The method of claim 12, wherein the arginine is L-arginine and the glycoside is administered as a component of TRUTINA DULCEM.

14. The method of claim 12, wherein the effective amount of the L-arginine is about 0.1 to about 60 g daily, or an equivalent molar quantity.

15. A method for treating a patient diagnosed with a complication associated with a condition resulting from dysregulated arginine metabolism, said method comprising: diagnosing the complication in a patient; and administering to the patient a composition comprising an effective amount of arginine and a glycoside compound.

16. The method of claim 15, wherein the arginine is L-arginine and the glycoside is administered as a component of TRUTINA DULCEM.

17. The method of claim 15, wherein the complication is one selected from the group consisting of: hypercholesterolemia; hyperlipidemia; hyperinsulinemia; dysglycemia; hyperuricemia; high triglyceride levels; obesity; cardiovascular disease; coronary artery disease; cardiac disease; pulmonary disease; vascular disease; hypertension; hyperglycemia; glucose intolerance; low high density lipoprotein levels; diabetes Types 1 and 2; arteriosclerosis; atherosclerosis; cerebrovascular thrombosis; cerebrovascular haemorrhage; stroke; angina; coronary thrombosis; coronary heart disease; intermittent claudication; and ischemia in the limbs.

18. The method of claim 15, wherein the effective amount of the L-arginine is about 0.1 to about 60 g daily, or an equivalent molar quantity.
